(19) United States
(12) Plant Patent Application Publication
(10) Pub. No.: US 2005/0198715 P1
(43) Pub. Date: Sep. 8, 2005

(54) Peach tree named 'S 6359'

(76) Inventor: Rene Francois Monet, Villenave D'Ornon (FR)

Correspondence Address: STRATTON BALLEW, 213 S 12TH AVE, YAKIMA, WA, 98902, US

(21) Appl. No.: 11/013,495

(22) Filed: Dec. 15, 2004

Related U.S. Application Data
(60) Provisional application No. 60/531,593, filed on Dec. 18, 2003.

(51) Int. Cl. A01H 5/00

(52) U.S. Cl. PLT/194

(57) ABSTRACT

A new cultivar of peach tree (Prunus persica L. Batsch) named ‘S-6359’ is disclosed. The new cultivar is notable for its large, yellow fleshed, high quality fruit.

### FIELD OF THE INVENTION

The invention relates to the field of peach trees. More specifically, the invention is a new and distinct cultivar of peach tree Prunus persica L. Batsch.

### BRIEF DESCRIPTION OF THE DRAWINGS

FIG. 1 shows a fruit of the new variety; and

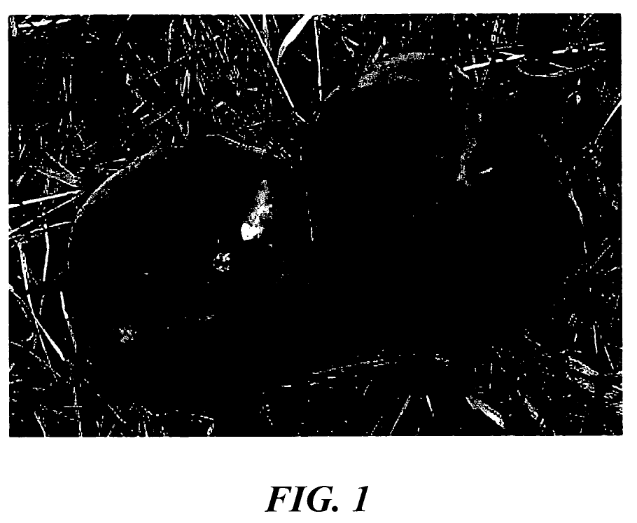

FIG. 2 shows a fruit of the new variety, including the flesh and stone thereof; and

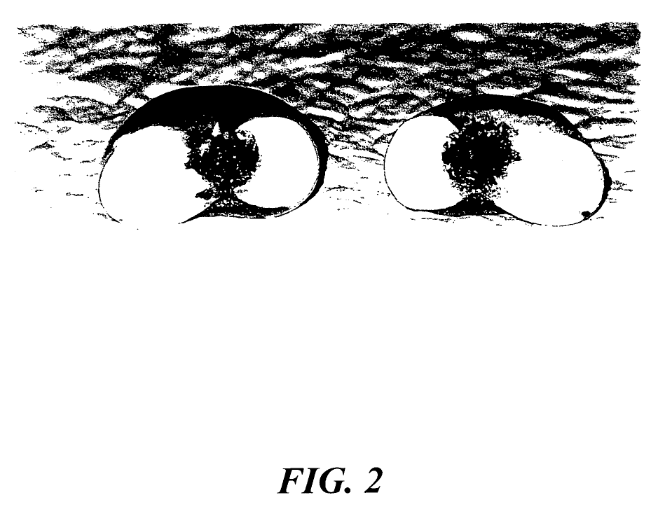

FIG. 3 shows a tree of the new variety.

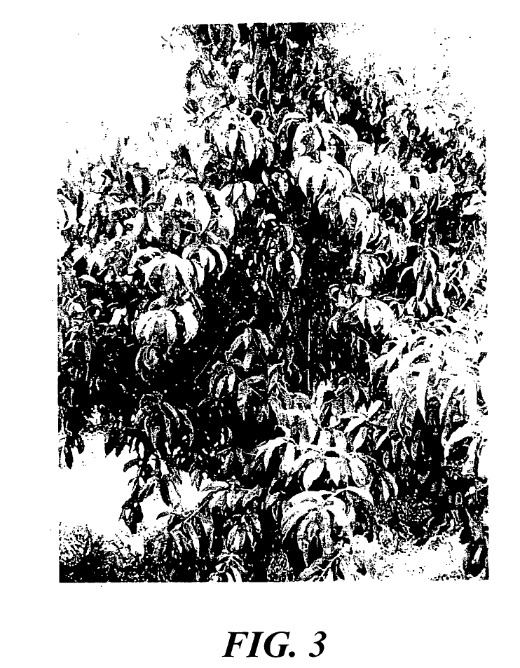

### BRIEF DESCRIPTION OF THE INVENTION

The new peach tree ‘S-6359’ was developed as part of a controlled breeding program. The new peach tree has been observed to remain true to type over successive asexually propagated generations.

1. We claim a new and distinct peach tree substantially as shown and described herein:
